(19) United States
(12) Patent Application Publication
(10) Pub. No.: US 2010/0261897 A1
(43) Pub. Date: Oct. 14, 2010

(54) Improved Process For the Preparation of Cephalosporin Antibiotic Intermediate

(75) Inventors: Udayampalayam Palanisamy Senthilkumar, Chennai (IN); Kanagaraj Suresh Kumar, Chennai (IN); Singaravel Mohan, Chennai (IN)

Correspondence Address: OLIFF & BERRIDGE, PLC, P.O. BOX 320850, ALEXANDRIA, VA, 22320-4850, US

(73) Assignee: ORCHID CHEMICALS & PHARMACEUTICALS LTD,, TAMIL NADU (IN)

(21) Appl. No.: 11/794,735

(22) PCT Filed: Jan. 16, 2006
(86) PCT No.: PCT/IB2006/000061

§ 371 Date: Jul. 5, 2007

(30) Foreign Application Priority Data
Jan. 17, 2005 (IN) 27/CHE/2005

(51) Int. Cl. C07D 501/18

(52) U.S. Cl. 540/219

(57) ABSTRACT

The present invention relates to a process for the production of cephalosporin antibiotic intermediate of formula (I). More particularly relates to the preparation of the compound of formula (I) using a solvent medium selected from the group consisting of decalin (decahydronapthalene), hexane, cyclohexene, tetralin, petroleum ether, wherein X represents HI, HCI, H2SO4 and the like. The compound of formula (I) is an important intermediate in the preparation of Cefepime.

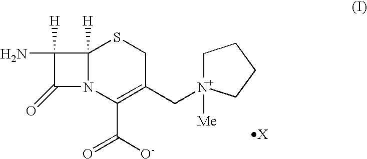

### FIELD OF THE INVENTION

The present invention relates to a process for the production of cephalosporin antibiotic intermediate of formula (I). More particularly relates to the preparation of the compound of formula (I).

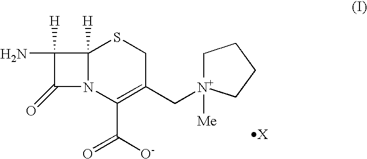

wherein X represents HI, HCl, H₂SO₄ and the like.

The compound of formula (I) is an important intermediate in the preparation of Cefepime.

### BACKGROUND OF THE INVENTION

Cefepime is chemically known as [6R-[6alpha,7beta(Z)]]-1-7-[(2-Amino-4-thiazolyl)(methoxyimino)acetylamino]-2-carboxy-8-oxo-5-thia-1-azabicyclo [4.2.0]oct-2-en-3-ylmethyl]-1-methylpyrrolidinium hydroxide inner salt or (6R,7R)-7-[2-(2-Aminothiazol-4-yl)-2(Z)-(methoxyimino)acetamido]-3-(1-methylpyrrolidiniomethyl)-3-cephem-4-carboxylate. Cefepime is a fourth-generation cephalosporin that is active against a wide range of gram-positive and gram-negative aerobic organisms and is disclosed in U.S. Pat. No. 4,406,899. According to this patent Cefepime is prepared by the following process:

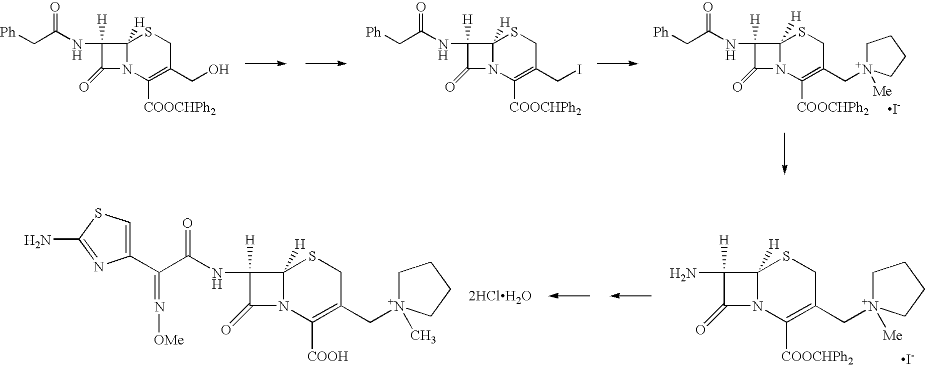

U.S. Pat. No. 4,868,294 claims a process for the preparation of stable, crystalline 7-amino-3-[(1-methyl-1-pyrrolidinio)methyl]ceph-3-em-4-carboxylate salts substantially free of the Δ² isomer starting from 7-amino cephalosporanic acid (7-ACA) in 1,1,2-trichlorotrifluoroethane (Freon TF) or 1,1,1-trichlorotrifluoroethane as the solvent as shown below:

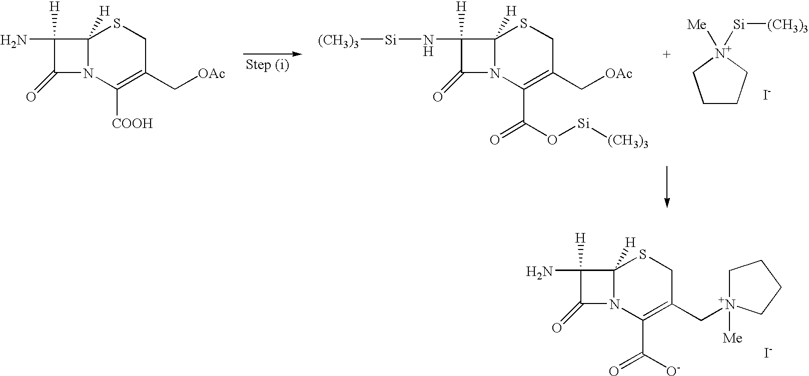

U.S. Pat. No. 5,594,531 claims almost a similar process for the preparation of stable, crystalline 7-amino-3-[(1-methyl-1-pyrrolidinio)methyl]ceph-3-em-4-carboxylate salts substantially free of the Δ² isomer by utilizing C₅₋₈ cycloalkanes as solvent.

During our continued search we have identified solvents, which yield the compound of formula (I) in good purity, which is substantially free from Δ² isomer and useful in the preparation of cefepime.

### OBJECTIVE OF THE INVENTION

The main objective of the present invention is to provide a process for the preparation of compound of formula (I) in good purity, which is substantially free from Δ² isomer.

Another objective of the present invention is to provide a process for the preparation of intermediate of formula (I), which is easy to implement on commercial scale.

### SUMMARY OF THE INVENTION

Accordingly, the present invention provide a process for the preparation of compound of formula (I)

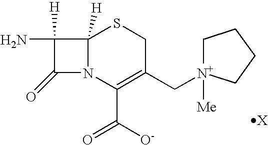

which comprises the steps of:
i) preparing solution A by reacting N-methylpyrrolidine of formula (IV) (NMP) with iodotrimethylsilane in a solvent consisting of hexanes, cyclohexene, decalin, tetralin or petroleum ether, or mixtures there of,
ii) preparing solution B by reacting 7-ACA of formula (II) with silylating agent in a solvent consisting of hexanes, cyclohexene, decalin (decahydronaphthalene), tetralin, petroleum ether, or mixtures there of,
iii) condensing solution A with solution B to produce compound of formula (VI), and
iv) treating the compound of formula (VI) with aqueous alcoholic solvent or alcoholic solvent or water and isolating the compound of formula (I).
The process is shown in Scheme-2

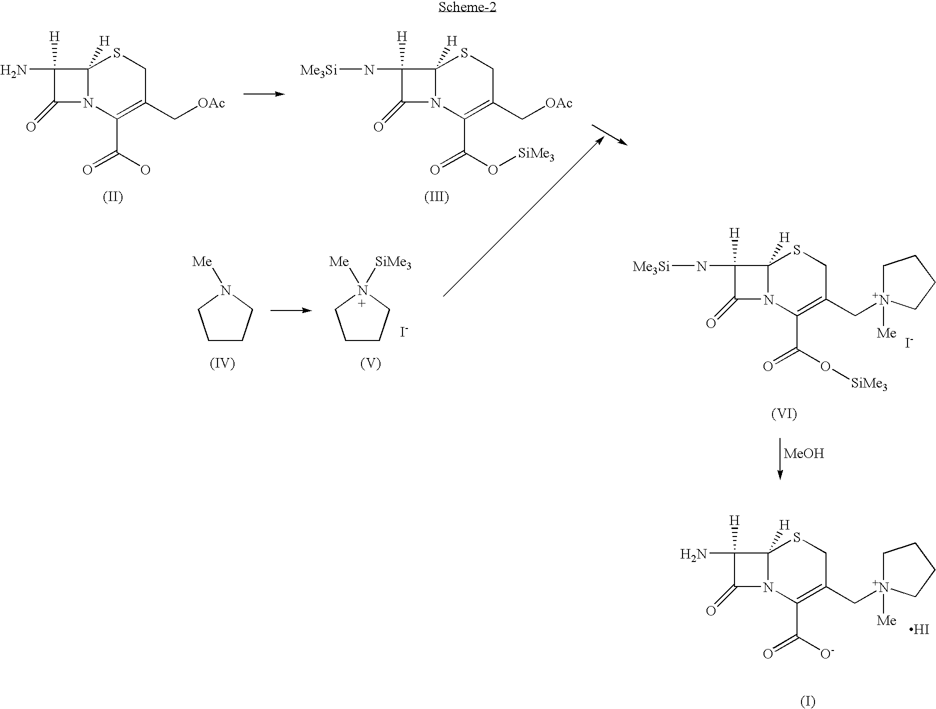

### DETAILED DESCRIPTION OF THE INVENTION

In an embodiment of the present invention silylating agent used in step (ii) is selected from hexamethyldisilazane (HMDS), trimethylchlorosilane(TMCS), trimethylsilyl iodide (TMSI), N,O-bis-(trimethylsilyl)-acetamide (BSA), methyltrimethylsilyltrifluoroacetamide (MSTFA), N,O-bis-(trimethylsilyl)trifluoroacetamide (BSTFA).

In another embodiment of the present invention iodotrimethylsilane is prepared by reacting hexamethyldisilane (HMD) with iodine at a temperature in the range of 10° C. to 100° C. in the presence of solvent selected from hexanes, cyclohexene, decalin, tetralin or petroleum ether. To this solution NMP was added to yield solution B.

In another embodiment of the present invention isolation of compound of formula (I) is carried out by reacting the compound of formula (VI) obtained in step (iii) with water or lower alkanol or aqueous lower alkanol such as methanol, isopropyl alcohol, butanol and the like.

In one more embodiment of the present invention the compound of formula (I) can be prepared by reacting silylated 7-ACA of formula (II) with N-methylpyrrolidine of formula (IV) in a solvent consisting of hexane, cyclohexene, decalin (decahydronapthalene), tetralin, petroleum ether or mixtures thereof to produce compound of formula (VI), followed by removing the silyl protecting group of formula (VI). The advantage of declain over cyclohexane is the solvent recovery is good in the case of declain, and hence economical from manufacturing point of view.

In yet another embodiment of the present invention the compound of formula (I) further converted to cefepime dihydrochloride monohydrate by the conventional method or by the method disclosed in our co-pending application No. 673/CHE/2003, 1020/CHENP/03, or 848/MAS/2002.

In still another embodiment of the present invention the compound of formula (I) can be prepared by utilizing the following scheme.

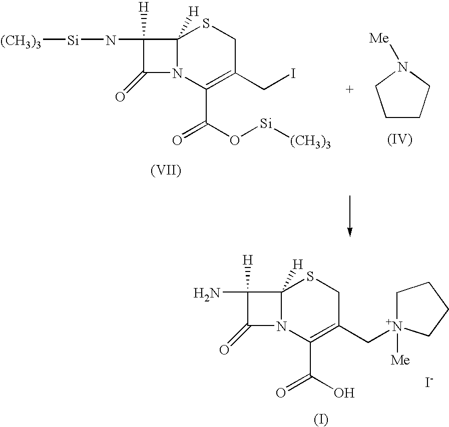

In yet another embodiment of the present invention, the compound of formula (VII) is prepared by reacting silylated 7-ACA of formula (III) with iodotrimethylsilane in the presence of solvent selected from hexanes, cyclohexene, decalin, tetralin or petroleum ether or mixtures thereof.

The present invention is provided by the examples below, which are provided by way of illustration only and should not be considered to limit the scope of the invention.

### Example 1

### (6R,7R)-7-Amino-3-[(1-methyl-1-pyrrolidino)methyl]ceph-3-em-4-carboxylate hydroiodide salt

### Solution A:

To a suspension of iodine [93.4 g 368 mmol] in hexanes (300 mL) at 20-30° C. was added hexamethyldisilane (HMD, 63.0 g, 430.4 mmol). The resulting suspension was heated to 60-70° C. and maintained for few hours. The above solution was cooled to 0° C. N-Methylpyrrolidine (26 g, 305.5 mmol) was added and the resultant slurry was stirred at 0-15° C.

### Solution B:

To a suspension of 7-ACA (50 g, 183.6 mmol) in hexanes (150 mL) was added BSA (78 g, 383.4 mmol) at 22-25° C. The resulting mixture was stirred at 30-35° C.

### Condensation:

The silylated 7-ACA solution [Solution B] was added to the Solution A at to 0° C. The suspension was heated to 34-37° C. and maintained till completion of reaction. After the completion of the reaction, reaction mixture was cooled to 3-5° C. Chilled water (100 mL, −50 to −55° C.) was added at 5-10° C. to de-silylate followed by/methanol and conc. HCl (30.4 mL). The temperature was allowed to raise 15° C. and stirred for 15 min. The aqueous phase was separated and organic phase was extracted with a 50% aqueous methanol (25.6 mL). To combined aqueous phase, methanol, sodium metabisulphite, EDTA and carbon, were added, stirred at 10-14° C. and filtered. The product was crystallized by adding triethylamine and adjusting the pH to 3.0-3.2 at 15-20° C. The slurry was cooled to −5 to 0° C. The solid obtained was filtered and washed with chilled 10% aqueous methanol followed by chilled methanol. Dried under vacuum at 35-40° C. to yield pure title compound.

### Example 2

### (6R,7R)-7-Amino-3-[(1-methyl-1-pyrrolidino)methyl]ceph-3-em-4-carboxylate hydroiodide salt

### Solution A:

To a suspension of iodine [93.4 g 368 mmol] in anhydrous decahydronaphthalene (300 mL) at 20-30° C. was added hexamethyldisilane (HMD, 63.0 g, 430.4 mmol). The resulting suspension was heated to 60-70° C. and maintained for few hours. The above solution was cooled to 0° C. N-Methylpyrrolidine (26 g, 305.5 mmol) was added and the resultant slurry was stirred at 0-15° C.

### Solution B:

To a suspension of 7-ACA (50 g, 183.6 mmol) in anhydrous decahydronaphthalene (150 mL) was added BSA (78 g, 383.4 mmol) at 22-25° C. The resulting mixture was stirred at 30-35° C.

### Condensation:

The silylated 7-ACA solution [Solution B] was added to the Solution A at 0° C. The suspension was heated to 34-37° C. and maintained till completion of reaction. After the completion of the reaction, reaction mixture was cooled to 3-5° C. Chilled 50% aqueous methanol (200 mL, −50 to −55° C.) was added at 5-10° C. followed by conc. HCl (30.4 mL). The temperature was allowed to raise 15° C. and stirred for 15 min The aqueous phase was separated and organic phase was extracted with a 50% aqueous methanol (25.6 mL). To combined aqueous phase, methanol, sodium metabisulphite (1 g) and carbon were added, stirred at 10-14° C. and filtered. The product was crystallized by adding triethylamine and adjusting the pH to 3.0-3.2 at 15-20° C.

The slurry was cooled to −5 to 0° C. The solid obtained was filtered and washed with chilled 10% aqueous methanol followed by chilled methanol. Dried under vacuum at 35-40° C. to yield pure title compound.

1. A process for the preparation of compound of formula (I) which comprises the steps of 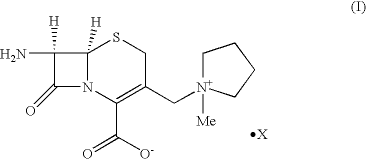
i) reacting silylated 7-ACA of formula (III) 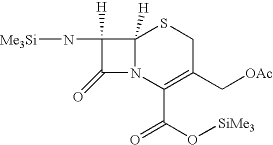
with N-methylpyrrolidine of formula (IV) 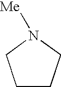
or its silylated derivative of formula (V) 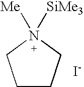
in a solvent medium consisting of hexane, cyclohexene, decalin (decahydronapthalene), tetralin, petroleum ether or mixtures thereof to produce compound of formula (VI), 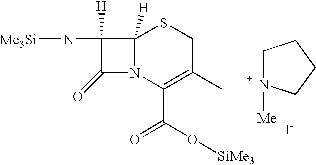
ii) treating the compound of formula (VI) with aqueous alcoholic solvent or alcoholic solvent or water and
iii) isolating the compound of formula (I).

2. A process as claimed in claim 1, wherein silylated 7-ACA of formula (III) prepared by reacting 7-ACA of formula (II) with silylating agent in a solvent consisting of hexanes, cyclohexene, decalin (decahydronaphthalene), tetralin, petroleum ether, or mixtures there of.

3. A process as claimed in claim 1, wherein silylated by reacting N-methylpyrrolidine of formula (IV) (NMP) with iodotrimethylsilane in a solvent consisting of hexanes, cyclohexene, decalin, tetralin or petroleum ether, or mixtures there of.

4. A process as claimed in claim 2, wherein the silylating agent is selected from hexamethyldisilazane (HMDS), trimethylchlorosilane (TMCS), trimethylsilyl iodide (TMSI), N,O-bis-(trimethylsilyl)-acetamide (BSA), methylltrimethylsilyltrifluoroacetamide (MSTFA), or N,O-bis-(trimethylsilyl)trifluoroacetamide (BSTFA).

5. A process as claimed in claim 1, wherein the alcoholic solvent employed for the isolation of compound of formula (I) in step (ii) is selected from methanol, ethanol, isopropyl alcohol, or butanol.

6. A process as claimed in claim 1, further comprising converting compound of formula (I) to cefepime dihydrochloride monohydrate by utilizing conventional technique.

7. A process as claimed in claim 3, wherein the silylating agent is selected from hexamethyldisilazane (HMDS), trimethylchlorosilane (TMCS), trimethylsilyl iodide (TMSI), N,O-bis-(trimethylsilyl)-acetamide (BSA), methylltrimethylsilyltrifluoroacetamide (MSTFA), or N,O-bis-(trimethylsilyl)trifluoroacetamide (BSTFA).
